(19) United States
(12) Patent Application Publication
(10) Pub. No.: US 2005/0276719 A1
(43) Pub. Date: Dec. 15, 2005

(54) Cleaning body composed of high-grade steels

(76) Inventor: Dirk Zielonka, Bielefeld (DE)

Correspondence Address: NIXON PEABODY, LLP, 401 9TH STREET, NW, SUITE 900, WASHINGTON, DC, 20004-2128, US

(21) Appl. No.: 11/199,082

(22) Filed: Aug. 9, 2005

Related U.S. Application Data
(62) Division of application No. 10/181,249, filed on Jul. 16, 2002.

(30) Foreign Application Priority Data
Jan. 17, 2000 (DE) 100 01 728.2

(51) Int. Cl. A61L 9/00

(52) U.S. Cl. 422/5

(57) ABSTRACT

The invention relates to a cleaning body made of solid high-grade steel for the cleaning of human skin under running water. The cleaning body has at least one cleaning-active area. A particularly effective odor-removing effect of the cleaning body is achieved by a cleaning-active area formed on the surface of the cleaning body by finally working the surface by metal-cutting.

### CROSS-REFERENCE TO RELATED APPLICATION

This application is a division of co-pending U.S. patent application Ser. No. 10/181,249.

### BACKGROUND OF THE INVENTION

1. Field of the Invention

The invention relates to a cleaning body made of high-grade steel for the cleaning of human skin, such as under running water. The cleaning body has at least one cleaning-active area formed by an accessible area of the surface of the cleaning body.

2. Description of Related Art

For hygienic and aesthetic reasons, all people regularly need to wash their hands. Especially after manual work, e.g., work on technical machines or work processing or preparing foodstuffs. After such work, visually detectable soiling of the hands often occurs, and is usually accompanied by a strong odor adhering to the soiled hands as well. The visually detectable soiling of the hands is removed by washing the hands with a conventional soap and/or with a washing paste, and, if necessary, with the additional aid of brushes. This sometimes leads to considerable irritation of the surface of the skin, especially when brushes are used. Despite this intensive cleaning treatment, which in some cases has a very irritating effect on the skin, it is often only possible to remove from the hands the soiling that is visible with the eye. Often the unpleasant odor remains. This odor often originates from substances such as lubricants and oils, e.g. gun oil, or certain foodstuffs, e.g., fish or garlic. It is nearly impossible to remove these odors with scented soaps without leaving residual traces, since the odors are merely masked by the scent of the soap for usually a short time.

The above problems of unpleasantly smelling hands do not occur solely in the areas of work or leisure, e.g., in the case of hunters and anglers who handle gun oil or come into contact with fish slime. This problem also arises in other quite everyday areas, e.g., among smokers who often complain about their fingers smelling strongly of nicotine. Another troublesome odor, even if not as unpleasant, can also be found for example on the hands of masseurs or hospital and nursing staff, who have to handle strong-smelling ointments and dressings and who wish to remove the odor concerned completely from their hands after they leave work.

### SUMMARY OF THE INVENTION

Accordingly, it is an object of the invention to provide a device for rapidly removing odors from the skin, particularly the hands, such that the skin is free from residual traces of the odor in a low-cost manner.

The cleaning body according to the invention is characterized by a cleaning-active area that is worked by metal-cutting in the last step of manufacture. The term “worked by metal-cutting” is understood here to mean that, in the production of the cleaning body, its cleaning-active area undergoes working by metal-cutting, such as for example by turning on a lathe, in the last, i.e. final, manufacturing step of the cleaning-active area surface. No further operating step occurs after this working by metal-cutting, and in particular, the cleaning-active surface does not thereafter undergo a finishing step, especially not a coating or polishing step.

The cleaning body according to the invention is used for the removal of odor from the hands in the same manner as a person would use conventional soap, i.e, the cleaning body is rubbed between the hands under running water for about 10 to 20 seconds.

Various high-grade steels can be used for the cleaning body according to the invention. Particularly good results with the removal of odor are achieved with cleaning bodies made of high-grade steel with material number 1.4301, and, in particular, a high-grade steel with material number 1.4571.

The surface of the cleaning-active area of the cleaning body according to the invention, having been finally worked by metal-cutting, is provided with particularly good properties for odor removal if the cleaning body is produced from solid material. This then ensures that the material structure of the high-grade steel is essentially in its original state, which provides for optimum removal of odor.

All sizes and shapes of cleaning bodies which can be handled easily and efficiently are in principle conceivable for the cleaning bodies according to the invention. In particular, sizes and shapes which are used for conventional washing soaps work well. A preferred embodiment of the cleaning body according to the invention, is a cleaning body formed in the shape of a circular or an oval disc. If the cleaning body according to the invention has the shape of a circular disc, it is particularly preferable for a cone-shaped section to be provided on at least one flat side of the circular disc, and particularly, preferably on both flat sides of the circular disc. Such a cone-shaped section facilitates handling of the cleaning body according to the invention, since that shape is particularly well adapted to the shape of the palm of the hand, which will result in a particularly intensive and large-area of skin contact to be achieved. Simply put, the larger and more efficient the skin contact of the cleaning body according to the invention, the better its effect of removing the odor. Accordingly, a particularly good effect of the cleaning body according to the invention is achieved when the cleaning body is designed in a lens-shape.

Specifically, when the cleaning body is designed in the shape of a disc or a lens, it has several edges. Such edges are not detrimental to the cleaning effect, i.e. the odor-removing effect of the cleaning body is not diminished. However, the effect of such edges is that the cleaning body according to the invention feels unpleasant in the hand and in the worst case can even lead to small injuries to the hand. In this regard, a preferred embodiment of the cleaning body according to the invention is in providing rounded off edges of the cleaning body. By doing this, the edges can also be gripped without discomfort.

In another embodiment, the cleaning body according to the invention can be composed of several parts. However, it is preferable for the cleaning body according to the invention to be designed in one piece.

The greatest cleaning effect of the cleaning body is achieved when as large a cleaning-active area as possible is provided. Therefore, according to an another embodiment of the cleaning body, provision is made such that a coating, an inscription, a glaze or a plastic cover is provided on an area separate from the cleaning-active area. In this way, the cleaning body can be provided with a manufacturer's or product name, so that its marketing as a branded product is facilitated. A glaze or a coating can be provided on aesthetic grounds in a separate area, or a plastic cover may be advantageous if placing of the metal surface on a sensitive base could cause damage to the latter.

According to another preferred embodiment of the cleaning body of the invention, provision is made such that the cleaning body has at least one recess or drilled hole. Although the overall area of the cleaning-active area is reduced, a weight-saving is achieved. It is particularly preferable for the recess or the drilled hole to be formed in an area outside the cleaning-active area.

In order that the cleaning body can be efficiently handled and a sufficient cleaning effect can be achieved, it is preferable for the length dimension of the cleaning body to be at least 2 cm, and more preferably at least 4 cm. In this way, a sufficiently large and long surface of the cleaning-active area is provided, and thus a sufficiently long path is available to permit the hand to be rubbed over this surface, which is essential for odor removal.

The invention further relates to a WC stone, in particular for odor-destroying or odor-reducing use in urinals.

The WC stone according to the invention is produced from high-grade steel and has at least one active area which is formed in an accessible area of its surface, whereby the active area is finally worked by metal-cutting.

The odor-destroying or odor-reducing effect of the WC stone according to the invention is based on the same principle as the effect of the cleaning body described above for the cleaning of human skin. Accordingly, the WC stone according to the invention is preferably produced from high-grade steel with material number 1.4571 or from high-grade steel with material number 1.4301. In this regard, it is also especially preferable for the WC stone to be produced from solid material.

In principle, the WC stone according to the invention can have similar shapes and sizes to the cleaning body according to the invention for the cleaning of human skin.

As with the cleaning body according to the invention for the cleaning of human skin, the WC stone according to the invention can also be composed of several parts, but preferably it is produced in one piece.

One of the main advantages of the WC stone according to the invention lies in the fact that it assumes precisely the same function as conventional WC stones made of soap, i.e. it has an odor-reducing or odor-destroying effect, but in contrast with the conventional WC stones made of soap it does not get used up. In principle, the WC stone according to the invention has an arbitrarily long life, and therefore does not need to be replaced.

The odor-reducing or odor-destroying function of the WC stone of the invention occurs when it is in contact with the ambient air and is rinsed round with water, and finds uses in other areas of application. In particular, an embodiment of the invention includes the use of a high-grade steel body in contact with the ambient air and with water for the purpose of odor-cleaning of the ambient air. The high-grade steel body has at least one cleaning-active area, which is formed on an air-accessible area of the surface of the high-grade steel body and which is finally worked by metal-cutting.

The invention further relates to a fish cosh. The fish cosh according to the invention is characterized in that it has a cleaning body as described above and a rod, preferably made of metal, as a handle, which is screwed into a thread provided in the cleaning body. An extremely practical tool with a twofold function is thus made available to fishermen and anglers. The fish cosh can be used to stupefy or chop off caught fish, and upon being provided with the cleaning body as a component of the fish cosh, the cosh can be used by the fisherman or angler to easily, efficiently and permanently wash off the smell of fish adhering to their hands. To do this, it is not necessary for the cleaning body to be unscrewed from the rod. In the unscrewed state, however, the cleaning body can also be used in the domestic kitchen, as a separate cleaning body.

The invention further relates to a knife, in particular an angler's or hunter's knife. The knife of the invention is characterized in that the handle of the knife is formed by a cleaning body as described above or the cleaning body is integrated into the handle of the knife. The knife, like the fish cosh described above, is an extremely practical tool for anglers or hunters. For example, a hunter carrying such a knife immediately has cleaning means available after gutting the game or after maintaining a weapon. The knife of the invention provides an efficient means to remove unpleasant odors permanently off the hands. As is apparent, such as in the case of the knife according to the invention, it is possible to have diverse shapes for the cleaning body that has been described above.

Consequently, there is a large number of variations for configuring and developing the invention. In this regard, reference is made to preferred examples which make reference to the drawings.

### BRIEF DESCRIPTION OF THE DRAWINGS

FIG. 1a shows, in a side view, a cleaning body according to one embodiment of the invention,

FIG. 1b shows, in an oblique view, the cleaning body according to FIG. 1a, and

FIG. 2 shows a fish cosh according to another embodiment of the invention.

### DETAILED DESCRIPTION OF THE INVENTION

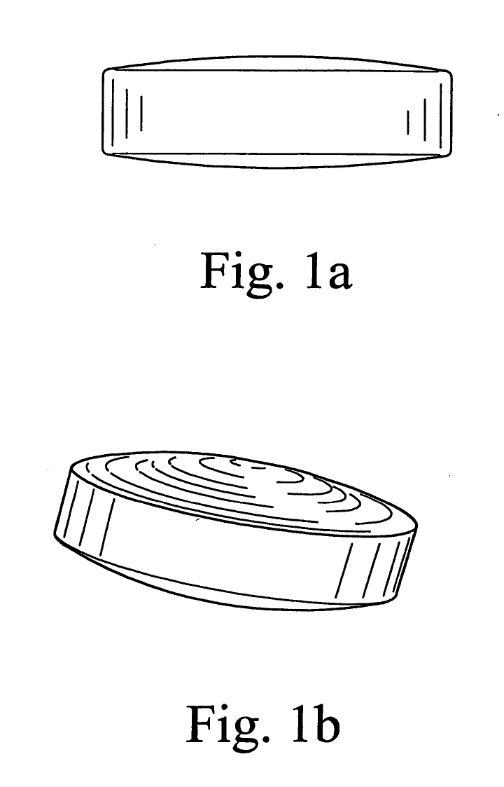

A cleaning body according to an embodiment of the invention is illustrated in FIG. 1. The cleaning body is turned from solid, high-grade steel with material number 1.4571. The cleaning body has a lens-shape and a diameter of approximately 49 mm. The thickness of the cleaning body at the outer edge is about 12 mm, and the thickness of the cleaning body in the center is about 16 mm.

Since the cleaning body according to the embodiment of the invention shown in FIG. 1 is turned from a rod of solid material, the whole surface of the cleaning body is finally worked by metal-cutting. Furthermore, no recesses or drilled holes are provided in the cleaning body according to this first embodiment of the invention: Therefore, the cleaning body has a substantial weight, but it can be held comfortably and offers a large cleaning-active area.

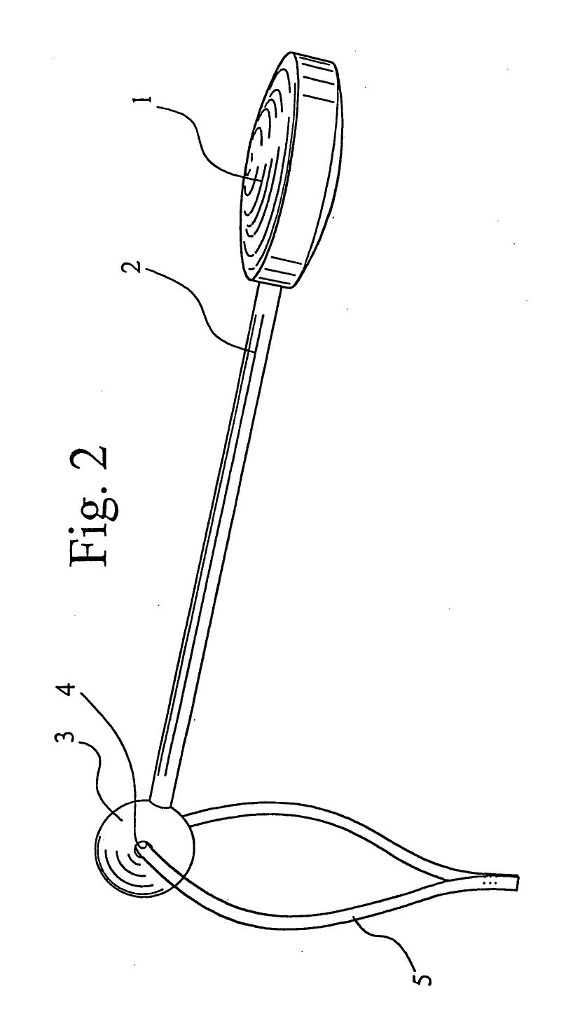

A fish cosh according to another embodiment of the invention is shown in FIG. 2 and is formed by the previously described cleaning body 1, which in a peripheral face is formed a radial M6 threaded hole into which a metal rod 2 is screwed. The metal rod is composed predominantly of brass and has a diameter of about 8 mm and a length of about 150 mm. Metals, other than brass, can of course also be used for rod 2. The cleaning body 1 screwed onto rod 2 is lens-shaped and has a diameter of approximately 49 mm. Its thickness is about 12 mm in its thinnest area, i.e. at the edge, and is about 16 mm in its thickest area, i.e. in the middle. Other dimensions are however also conceivable which, as with conventional fishing coshes, are sized according to the size of the expected fish.

On end of the rod 2 that is not screwed into cleaning body 1, the rod 2 has a plastic ball 3. The plastic ball 3 is also screwed into rod 2 by an M6 thread which is provided in plastic ball 3. As can also be seen from FIG. 2, a drilled hole 4 is also provided in plastic ball 3 at right angles to the longitudinal direction of rod 2, through which a hand strap 5 is passed. When the fish cosh is being used, the latter can be placed around the wrist and thus prevents the fish cosh from being lost if it inadvertently slips out of the hand during use.

1. A method for removing odors from ambient air comprising:
contacting a cleaning body composed of high grade steel with ambient air and water;
wherein the cleaning body includes at least one active, oder removing area formed in an accessible area of a surface of the cleaning body, and wherein the active, oder removing area is finally worked by metal-cutting.

2. Method according to claim 1, wherein the cleaning body is used as a toilet stone.
